(19) United States
(12) Patent Application Publication
(10) Pub. No.: US 2023/0089899 A1
(43) Pub. Date: Mar. 23, 2023

(54) METHOD FOR NON-TARGETED CHARACTERISATION OF A SOLUTION COMPRISING A PLURALITY OF SOLUTES

(71) Applicant: COMMISSARIAT A L'ENERGIE ATOMIQUE ET AUX ENERGIES ALTERNATIVES, Paris (FR)

(72) Inventors: Romain Dagnelie, Massy (FR); Zoé Disdier, Saint-Cloud (FR)

(21) Appl. No.: 17/931,574

(22) Filed: Sep. 13, 2022

(30) Foreign Application Priority Data
Sep. 17, 2021 (FR) 2109802

(51) Int. Cl. G16C 20/30

(52) U.S. Cl. CPC G16C 20/30

(57) ABSTRACT

A method for characterising a solution S₀ comprising a plurality of solutes, which comprises at least the steps consisting in: subjecting the solution S₀ to a sequence of N successive rows of liquid-liquid extractions, this sequence leading to a total of [2^{(N+1)}−1] different phases, 2^{N} phases of which are issued from the last row N of partitions; acquiring experimental data by measuring an extensive and conservative quantity X in the 2^{N} phases issued from the last row N of partitions of said sequence; and subjecting the experimental data to mathematical processing allowing to determine the distribution of the solutes of the solution S₀, which contribute to the values of the quantity X obtained experimentally, according to various hydrophilicity/lipophilicity values.

### CROSS-REFERENCE TO RELATED APPLICATIONS

This application claims priority from French Patent Application No. 2109802 filed on Sep. 17, 2021. The content of this application is incorporated herein by reference in its entirety.

### TECHNICAL FIELD

The invention relates to the field of characterisation of solutions.

More specifically, it relates to a method for non-targeted characterisation of a solution comprising a plurality of solutes which is based on a sequence of partitions, or extractions, of these solutes between two immiscible liquid phases, respectively aqueous and organic.

This method has a use in all the fields in which it can be desired to characterise an aqueous or organic solution without having to determine the composition thereof.

Thus, it can in particular have a use for:—

characterising an agricultural or industrial effluent with a view to its later management, in particular an effluent having a complex composition comprising, for example, solutes issued from a biotic or abiotic (chemical, radiolytic or other) degradation;

evaluating the quality of a natural or industrial product, in particular of a food nature (and, thereby, the reliability of the methods implemented for its culture, its collection or its manufacturing, its preservation and its distribution);

evaluating the quality of a water such as a water issued from a wastewater treatment plant (and, thereby, the reliability of the treatment method used), a seawater or the water from a waterway, a body of water, natural or artificial, or from a phreatic zone; or

evaluating the degree of purity of synthetic substances, marked or not, and characterising the quantity and the properties of the possible impurities that these substances comprise.

### PRIOR ART

There are numerous methods allowing to characterise a solution.

Among these methods, a difference is made between “direct” or “targeted” methods and “non-targeted” methods.

As their name indicates, the targeted methods aim to detect the presence of known solutes in the solution and, generally, to determine the concentration thereof. These methods typically comprise a previous step of separation of the solutes, for example, by chromatography.

Although applied in numerous fields, including, in particular, the monitoring and the surveillance of natural environments, the targeted methods are of limited interest when the solution that must be characterised has a complex composition and/or comprises unknown solutes, which is in particular the case of solutions comprising solutes resulting from a bacterial, chemical, radiolytic or other degradation of organic compounds.

This is why non-targeted methods have been proposed that aim to detect the presence in a solution of a range of solutes as broad as possible, including solutes never studied and, therefore, still unknown. Examples of non-targeted methods include that which is based on the use of liquid chromatography coupled with high-resolution mass spectrometry (or LC-HRMS) and which was in particular implemented by Mondeguer et al. (Spectra Analyse 2012, 284, 25-33) to establish a profiling of the metabolomic type of the bioactive toxins of the phytoplankton that accumulate in shells, and by Fischer et al. (Trends in Analytical Chemistry 2021, 136, 116188) to monitor the presence of pesticides in foods.

Although certain non-targeted methods have arrived at a stage of development that allows them to have a validation or an accreditation, they generally remain difficult to implement and insufficient when solutions having a particularly complex unknown composition must be characterised.

### DISCLOSURE OF THE INVENTION

The invention aims precisely to propose a method for non-targeted characterisation of a solution—called solution S₀ hereinafter—which is simple to implement and which can be applied to any solution, aqueous or organic, having an unknown composition, regardless of the complexity of this composition.

This method comprises at least the steps consisting in:

subjecting the solution S₀ to a sequence of N rows i of partitions, N being greater than or equal to 2 and i ranging from 1 to N, the rows i of partitions comprising:

for i=1, the mixture of a sample of the solution S₀ with a volume of an organic S_{org} or aqueous S_{aq} solution immiscible with the solution S₀ to obtain, after separation of the mixture into two phases, an aqueous phase φ(i=1,aq) and an organic phase φ(i=1,org); and

for i=2 to N:
the mixture of each aqueous phase φ(i−1,aq) with a volume of the solution S_{org} to obtain, after separation of the mixture into two phases, an aqueous phase φ(i,aq) and an organic phase φ(i,org);
the mixture of each organic phase φ(i−1,org) with a volume of the solution S_{aq} to obtain, after separation of the mixture into two phases, an aqueous phase φ(i,aq) and an organic phase φ(i,org);

b) subjecting all the aqueous and organic phases obtained in the row N to a measurement of an extensive and conservative quantity X to obtain an experimental vector \vec{V}exp comprising 2^{N} measured values of X;

c) calculating theoretical vectors \vec{V}theor(Dₖ) for y partition coefficient values Dₖ with y greater than or equal to 2 and k ranging from 1 to y, each theoretical vector comprising 2^{N} values of X calculated for the aqueous and organic phases obtained in the row N and for one of the y values Dₖ; and

d) carrying out a parametric adjustment between the vectors \vec{V}theor(Dₖ) and the vector \vec{V}exp to obtain a distribution of the solutes of the solution S₀, which contribute to the 2^{N} measured values of X, as a function of to the y values Dₖ.

Thus, the method of the invention is based on:

carrying out a sequence of N successive rows of liquid-liquid extractions, this sequence leading to a total of [2^{(N+1)}−1] different phases, 2^{N} phases of which come from the last row N of partitions (step a) of the method),

acquiring experimental data by measuring a quantity X in the 2^{N} phases issued from the last row N of partitions of said sequence (step b) of the method), and

mathematical processing of the experimental data thus acquired, this processing allowing to determine the distribution of the solutes of the solution S₀, which contribute to the values of the quantity X obtained experimentally, according to various hydrophilicity/lipophilicity values (steps c) and d) of the method).

Given that the hydrophilic/lipophilic nature of solutes is intrinsic to these solutes and represents a relevant piece of information for a certain number of their properties such as, for example, their solubility in a medium such as the environment and, therefore, their becoming in this medium, their toxicity, their bioaccumulation, etc., the method allows to obtain a useful piece of information on the solution S₀ but without having to determine the exact composition thereof or identifying the solutes that it comprises.

According to the invention, N is typically between 2 and 20 and, preferably, between 3 and 10.

The solution S₀ is, preferably, an aqueous solution, in which case the solution S_{org} is typically an organic solvent immiscible in water, optionally previously hydrated (that is to say pre-balanced by contact with water), while the solution S_{aq} is an aqueous solution that has, preferably, an ionic strength and a pH close to those of the solution S₀ and which can thus comprise, to do this, a ground salt of the type KCl or NaClO₄ and/or a pH buffer in suitable proportions in water, preferably distilled.

The organic solvent immiscible in water is, for example, a C8 to C12 alcohol (such as n-octanol, n-decanol or n-dodecanol), a C5 to C16 alkane (such as n-hexane, n-heptane or n-octane), cyclohexane, an aromatic hydrocarbon (such as benzene, toluene, ethylbenzene or xylene), chloroform, etc. Among these, preference is given to n-octanol.

Alternatively, the solution S₀ can also be an organic solution, in which case the solution S_{aq} is typically water, preferably distilled, while the solution S_{org} can be the same organic solvent as that the solution S₀ comprises in the case in which it is known what the organic solvent of the solution S₀ is, or, otherwise, one of the organic solvents mentioned above, optionally previously hydrated, preference being given, here also, to n-octanol.

According to the invention, the quantity X can be any quantity allowing to target a property of interest that the solutes or certain solutes of the solution S₀ have as long as this quantity is extensive and conservative.

“Extensive” quantity means a quantity, the sum of the values of which for two disjointed systems is equal to the value of the quantity for the combination of the two systems, which means concretely, in the context of the method of the invention, that, for a phase having a given composition, the quantity X is proportional to the volume of this phase.

“Conservative” quantity means a quantity that remains constant for an isolated system not exchanging material with the outside, which means concretely, in the context of the method of the invention, that the quantity X is preserved at each partition and that in the absence of any sampling of material, the sum of the values of X in the aqueous and organic phases obtained in the row N of partitions is equal to the value of X in the solution S₀.

Thus, the quantity X can in particular be an amount of substance (molar, by weight or other), a concentration (if the amount of substance is relative to a constant unit of volume), an absorbance, a fluorescence, a radioactivity or an opacity.

According to the invention, each vector \vec{V}theor(Dₖ) can be defined by applying:

to the partition of the sample of the solution S₀, the following equations (1a) and (1b):

$${X}_{( i = 1 , aq )} ( {D}_{k} ) = {X}_{0} ⨯ \frac{1}{( 1 + {D}_{k} ⨯ \frac{{V}_{( 0 , org )}}{{V}_{( 0 , a q )}} )} \qquad ( 1 a )$$

$${X}_{( i = 1 , org )} ( {D}_{k} ) = {X}_{0} ⨯ \frac{{D}_{k} ⨯ \frac{{V}_{( 0 , org )}}{{V}_{( 0 , aq} )}}{( 1 + {D}_{k} ⨯ \frac{{V}_{( 0 , org )}}{{V}_{( 0 , aq )}} )} \qquad ( 1 b )$$

to the partitions of the phases φ(i,aq), the following equations (2a) and (2b):

$${X}_{( i + 1 , aq )} ( {D}_{k} ) = {X}_{( i , aq )} ( {D}_{k} ) ⨯ \frac{1}{( 1 + {D}_{k} ⨯ \frac{{V}_{( i , org )}}{{V}_{( i , aq )}} )} \qquad ( 2 a )$$

$${X}_{( i + 1 , org )} ( {D}_{k} ) = {X}_{( i , aq )} ( {D}_{k} ) ⨯ \frac{{D}_{k} ⨯ \frac{{V}_{( i , org )}}{{V}_{( i , aq} )}}{( 1 + {D}_{k} ⨯ \frac{{V}_{( i , org )}}{{V}_{( i , aq )}} )} \qquad ( 2 b )$$

to the partitions of the phases φ(i,org), the following equations (3a) and (3b):

$${X}_{( i + 1 , aq )} ( {D}_{k} ) = {X}_{( i , org )} ( {D}_{k} ) ⨯ \frac{1}{( 1 + {D}_{k} ⨯ \frac{{V}_{( i , org )}}{{V}_{( i , aq )}} )} \qquad ( 3 a )$$

$${X}_{( i + 1 , org )} ( {D}_{k} ) = {X}_{( i , org )} ( {D}_{k} ) ⨯ \frac{{D}_{k} ⨯ \frac{{V}_{( i , org )}}{{V}_{( i , aq} )}}{( 1 + {D}_{k} ⨯ \frac{{V}_{( i , org )}}{{V}_{( i , aq )}} )} \qquad ( 3 b )$$

where:

X_{(i=1,aq)} is the expected value of X for solutes having the value Dₖ and for the phase φ(i=1,aq),

X_{(i=1,org)} is the expected value of X for solutes having the value Dₖ and for the phase φ(i=1,org),

X₀ is the measured value of X in the solution S₀,

V_{(0,org)} is the volume of the sample of solution S₀ if the solution S₀ is an organic solution or the volume of solution S_{org} used to partition the sample of the solution S₀ if the solution S₀ is an aqueous solution,

V_{(0,aq)} is the volume of the sample of solution S₀ if the solution S₀ is an aqueous solution or the volume of solution S_{aq} used to partition the sample of the solution S₀ if the solution S₀ is an organic solution,

X_{(i+1,aq)} is the expected value of X for solutes having the value Dₖ and for a phase φ(i+1,aq) issued from the partition of a phase φ(i,aq) or of a phase φ(i,org),

X_{(i+1,org)} is the expected value of X for solutes having the value Dₖ and for a phase φ(i+1,org) issued from the partition of a phase φ(i,aq) or of a phase φ(i,org),

X_{(i,aq)} is the expected value of X for solutes having the value Dₖ and for the partitioned phase φ(i,aq),

X_{(i,org)} is the expected value of X for solutes having the value Dₖ and for the partitioned phase φ(i,org),

V(i,aq) is the volume of the partitioned phase φ(i,aq) or the volume of solution S_{aq} used to partition the phase φ(i,org),

V(i,org) is the volume of the partitioned phase φ(i,org) or the volume of solution S_{org} used to partition the phase φ(i,aq),

whereby, for the aqueous and organic phases of the row N, 2^{N} expected values of X are obtained for solutes having the value Dₖ. These 2^{N} extensive values of X form a vector \vec{V}théor(Dₖ).

The parametric adjustment involves, preferably, determining a set of percentages Pₖ as weighting coefficients such that the sum of the vectors \vec{V}theor(Dₖ) weighted by these weighting coefficients is as close as possible to the vector \vec{V}exp or, in other words, such that:

\vec{V}exp≈Σ(Pₖ(%)×\vec{V}théor(Dₖ)).

This adjustment can be carried out by a conventional regression method such as the method of least squares, via software of the type Excel™ or OriginLab™.

The adjustment can further comprise an adjustment of the values of D and, therefore, of the vectors \vec{V}theor(Dₖ) themselves. The result of the adjustment is, for example, expressed in the form of a histogram representing, in the form of percentages (Pₖ), the distribution of the solutes of the solution S₀, which contribute to the 2^{N} measured values of X, as a function of the y partition coefficient values (Dₖ).

According to the invention, the solutes are, preferably, organic solutes.

Other features and advantages of the invention will appear upon reading the supplemental description that follows and that is given in reference to the appended figures.

It goes without saying that this supplemental description is only given as an illustration of the object of the invention and in no case forms a limitation of this object.

### BRIEF DESCRIPTION OF THE FIGURES

FIG. 1A and FIG. 1B illustrate, schematically and as an example, the usefulness of subjecting two solutions 1 and 2 having different compositions to a sequence of N rows of partitions for their characterisation and, thus, the highlighting of their difference in composition.

FIG. 2 illustrates, schematically and as an example, the phases that are obtained in the case in which a solution S₀ is subjected to a sequence of 3 rows of partitions.

FIG. 3 illustrates, schematically and as an example, a mathematical processing of the experimental data and of the result obtained by this mathematical processing.

FIG. 4 illustrates, schematically and as an example, the results of an experiment that was carried out with a known mixture of three solutes and allowed to validate the method of the invention.

FIG. 5 illustrates, as an example, the results of applying the method of the invention to the evaluation of the degree of purity of a commercial substance, namely bisphenol A marked with ¹⁴C, and to the characterisation of the quantity and of the lipophilicity of an impurity that this substance has.

### DETAILED DISCLOSURE OF PREFERRED EMBODIMENTS

### Reminders of Theory

The partition coefficient of a solute A expresses the distribution of this solute between two immiscible phases, respectively aqueous and organic, that are placed in contact with one another.

It is typically defined by a ratio of concentrations, noted below as D(A), according to the equation (4):

$$D ( A ) = \frac{{[ A ]}_{org}}{{[ A ]}_{a q}} \qquad ( 4 )$$

where:

[A]_{org} is the molar concentration of the solute A in the organic phase at equilibrium, and

[A]_{aq} is the molar concentration of the solute A in the aqueous phase at equilibrium.

For a solute A that appears in solution as several species (such as a weak acid that is dissociated into a base A⁻ and a proton), the partition coefficient D(A) is defined by the equation (5):

$$D ( A ) = \frac{{Σ [ {A}_{i} ]}_{org}}{{Σ [ {A}_{i} ]}_{a q}} \qquad ( 5 )$$

where:

[Aᵢ]_{org} is the molar concentration of each species of the solute A in the organic phase at equilibrium,

[Aᵢ]_{aq} is the molar concentration of each species of the solute A in the aqueous phase at equilibrium.

It is also possible to define the partition coefficient by a ratio in numbers of moles, noted below as D′(A), according to the equation (6):

$${D}^{′} ( A ) = \frac{{n ( A )}_{org}}{{n ( A )}_{a q}} = D ( A ) \frac{{V}_{org}}{{V}_{a q}} \qquad ( 6 )$$

where:

n(A)_{org} is the number of moles of the solute A present in the organic phase at equilibrium,

n(A)_{aq} is the number of moles of the solute A present in the aqueous phase at equilibrium,

V_{org} is the volume of the organic phase, and

V_{aq} is the volume of the aqueous phase.

For a solute A that appears in solution as several species, the equation (6) becomes the equation (7):

$${D}^{′} ( A ) = \frac{Σ {n ( {A}_{i} )}_{org}}{Σ {n ( {A}_{i} )}_{a q}} \qquad ( 7 )$$

where:

n(Aᵢ)_{org} is the number of moles of each species of the solute A present in the organic phase at equilibrium, and

n(Aᵢ)_{aq} is the number of moles of each species of the solute A present in the aqueous phase at equilibrium.

It should be noted that the partition coefficients D and D′ do not have a dimension and are generally expressed in the form of their logarithm to the base ten, respectively noted below as log D and log D′.

The partition of a solute A in solution between two phases, respectively aqueous and organic, leads to the following outcomes (8a), (8b), (9a) and (9b):

$${C}_{a q} = {C}_{0} ⨯ \frac{1}{( 1 + D )} \qquad ( 8 a )$$

$${C}_{org} = {C}_{0} ⨯ \frac{D}{( 1 + D )} \qquad ( 8 b )$$

$${n}_{a q} = {n}_{0} ⨯ \frac{1}{( 1 + {D}^{′} )} \qquad ( 9 a )$$

$${n}_{org} = {n}_{0} ⨯ \frac{{D}^{′}}{( 1 + {D}^{′} )} \qquad ( 9 b )$$

where:

C₀ is the initial concentration of the solute A (or the sum of the initial concentrations of its species) in the solution subjected to the partition,

C_{aq} is the concentration of the solute A (or the sum of the concentrations of its species) in the aqueous phase issued from the partition,

C_{org} is the concentration of the solute A (or the sum of the concentrations of its species) in the organic phase issued from the partition,

n₀ is the number of moles of the solute A (or the sum of the moles of its species) initially present in the solution subjected to the partition,

n_{aq} is the number of moles of the solute A (or the sum of the moles of its species) present in the aqueous phase issued from the partition, and

n_{org} is the number of moles of the solute A (or the sum of the moles of its species) present in the organic phase issued from the partition.

### 2. Usefulness of a Sequence of N Rows of Partitions and Application to the Method of the Invention (Step a) of the Method)

When a solution comprising several solutes is subjected to a single row of partitions, the measurement of a quantity X, such as a concentration, in the aqueous and organic phases issued from the first row of partitions provides a weighted average of the partition coefficient of each of the solutes contributing to this quantity.

Consequently, for a given quantity X, it is possible to obtain identical partition coefficients for solutions having different compositions if these solutions are subjected to a single row of partitions.

However, if these solutions are subjected to a second row of partitions, then the partition coefficients obtained for the phases issued from the second row of partitions allow to differentiate the solutions.

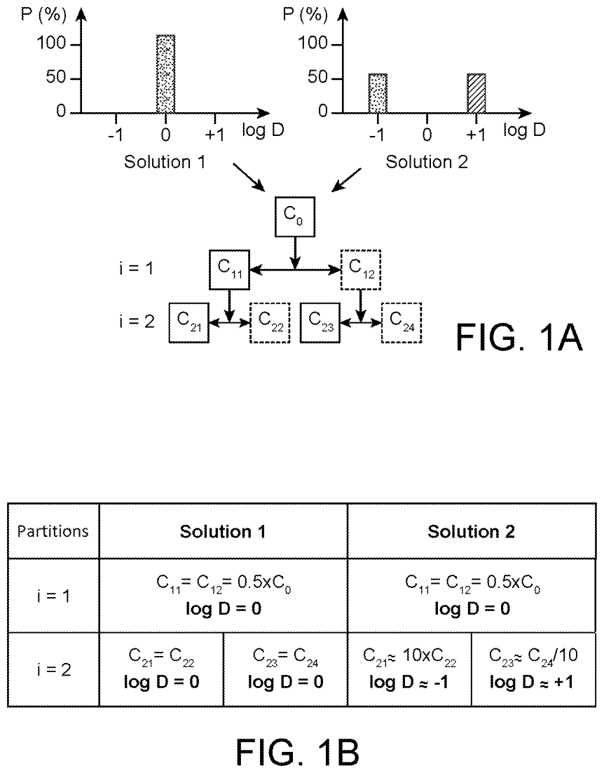

This is illustrated in FIGS. 1A and 1B for two solutions, one of which—called solution 1—only comprises one solute which is as hydrophilic as lipophilic (log D=0) whereas the other—called solution 2—comprises two solutes in equimolar quantities, one of which is hydrophilic (log D=−1) and the other of which is lipophilic (log D=+1), and which are subjected to two rows of partitions, each partition being carried out with identical volumes, noted as V, of aqueous and organic solutions.

In FIGS. 1A and 1B:

C₀ corresponds to the total concentration of solute(s) measured in the solutions 1 and 2, and the extensive quantity X would thus be n₀=C₀×V.

C₁₁ and C₁₂ correspond respectively to the total concentrations in the two phases, respectively aqueous and organic, issued from the first row of partitions, noted as i=1, of the solutions 1 and 2.

C₂₁ and C₂₃ correspond to the total concentrations in the two aqueous phases issued from the second row of partitions, noted as i=2, while

C₂₂ and C₂₄ correspond to the total concentrations in the two organic phases issued from the second row of partitions.

As visible in FIG. 1B, the solutes of solution 2 are distributed between aqueous phase and organic phase differently from the solute of solution 1 during the first row of partitions. Nevertheless, their respective contributions to the quantity X “balance themselves” (C₁₁=C₁₂=0.5×C₀) so that in the case of solution 2, an average log D of 0 and, therefore, equal to that obtained for solution 1 is obtained. The solutions 1 and 2 cannot therefore be differentiated.

However, the logs D obtained for the phases issued from the second row of partitions of solution 2, which are respectively equal to −1 and +1, allow to differentiate this solution from solution 1, the log D of which is equal to O.

It is this principle that is put to good use in the method of the invention according to which the solution S₀ that it is sought to characterise is subjected to a sequence of N rows of partitions, N being an integer greater than or equal to 2.

The first row of partitions corresponds to the partition of a sample of the solution S₀ that is carried out by mixing this sample with a volume of a solution immiscible with the solution S₀ in order for the solutes present in the solution S₀ to be distributed between the two phases forming the mixture, then, once the solutes are at equilibrium, by separating the two phases of the mixture from one another, for example by decantation or centrifugation.

If the solution S₀ is an aqueous solution, then the solution immiscible with it is an organic solution, typically an organic solvent immiscible in water, for example n-octanol (that has been, preferably, previously hydrated, that is to say pre-balanced by contact with water).

If the solution S₀ is an organic solution, then the solution immiscible with it is an aqueous solution, typically water, preferably distilled.

After the first row of partitions, two phases, respectively aqueous and organic, are thus obtained.

Each row of partitions after the first row of partitions corresponds to the partitions of the phases issued from the row of partitions that precedes it, partitions that are carried out:

by mixing each aqueous phase with a volume of an organic solution then, once the solutes are at equilibrium, by separating the mixture thus obtained into two phases, respectively aqueous and organic, for example by decantation or centrifugation, and

by mixing each organic phase with a volume of an aqueous solution then, once the solutes are at equilibrium, by separating the mixture thus obtained into two phases, respectively aqueous and organic, for example by decantation or centrifugation.

If the solution S₀ is an aqueous solution, then:

the organic solution, which is used in each of the rows of partitions after the first row of partitions, is the same as that which is used in the first row of partitions, whereas

the aqueous solution, which is used in each of the rows of partitions after the first row of partitions, is an aqueous solution that has, preferably, an ionic strength and a pH close to those of the aqueous solution S₀ and that comprises, if necessary, a ground salt such as KCl or NaClO₄ and/or a pH buffer in water, preferably distilled.

If the solution S₀ is an organic solution, then:

the aqueous solution, which is used in each of the rows of partitions after the first row of partitions, is the same as that used in the first row of partitions, whereas

the organic solution, which is used in each of the rows of partitions after the first row of partitions, is either the same organic solvent as that which the solution S₀ comprises or, otherwise, an organic solvent immiscible in water, optionally previously hydrated, such as n-octanol.

A total of [2^{(N+1)}−1] phases is thus obtained (counting the solution S₀ that it is sought to characterise), 2^{N} phases of which come from the last row of partitions.

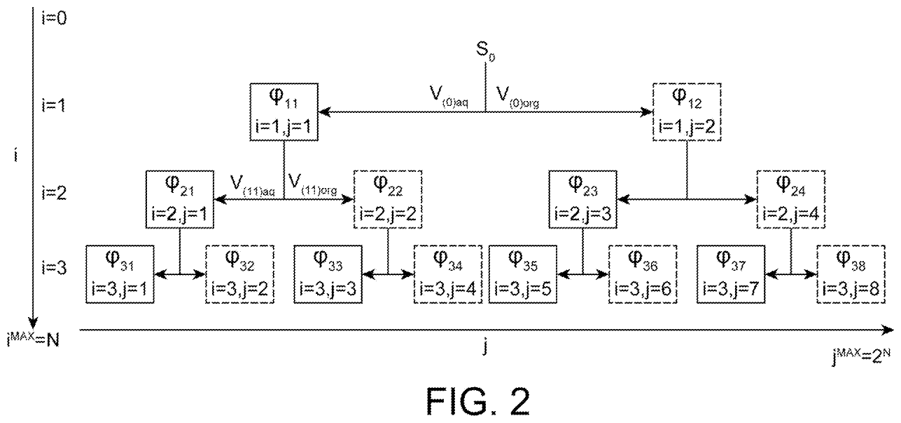

For example, FIG. 2 schematically illustrates the phases obtained in the case of a sequence of 3 rows of partitions (N=3).

In this figure, the phases inscribed in rectangles, the contours of which are solid lines correspond to aqueous phases while the phases inscribed in rectangles, the contours of which are dotted lines correspond to organic phases, and the solution S₀ can indifferently be aqueous or organic.

Moreover, in this figure, all the phases are assigned an index with two digits, the first digit of which corresponds to the row of partitions, noted as i, from which they come and the second digit of which corresponds to the order, noted as j, in which they lie in this row of partitions.

Thus, for example, the phase φ₃₄ is the 4th phase (j=4) issued from the 3^{rd} row (i=3) of partitions.

It goes without saying that the greater N, the finer the characterisation of the solution S₀, N being limited, however, by the sensitivity of the technique used to measure the quantity X since the values of the quantity X decrease as the number of rows of partitions increases.

This is why N is typically between 2 and 20 and, preferably, between 3 and 10.

### 3. Measurements of the Quantity X (Step b) of the Method)

As mentioned above, according to the method of the invention, a quantity X is measured in all the phases, aqueous and organic, obtained in the last partition row N.

The quantity X can be any quantity allowing to target a property of interest that the solutes or some of the solutes of the solution S₀ have as long as this quantity is extensive on the one hand and conservative on the other hand, that is to say that:

^{j}Σx₍ᵢⱼ₎=^{j}EX_{(i+1,j)}.

Thus, this can in particular be:

an amount of substance (molar, by weight or other) or a concentration (relative to an amount of substance by multiplying by the volumes of the solution S₀ or of the phases measured) such as total organic carbon (TOC), total nitrogen (according to the Kjeldahl method), chemical oxygen demand (COD), biochemical oxygen demand (BOD), suspended solids (SS), etc.,

an absorbance at a given wavelength,

a fluorescence at a given excitation or emission wavelength,

a radioactivity (such as the activity of the ¹⁴C for example),

a content of colloids, or

an opacity.

2^{N} measured values of X are thus obtained that allow to define an experimental vector \vec{V}exp which, in the case, for example, of the sequence of partitions shown in FIG. 2 has 8 values, respectively X₃₁, X₃₂, X₃₃, X₃₄, X₃₅, X₃₆, X₃₇ and X₃₈, each value of X being assigned the same index as that affecting the phase, aqueous or organic, for which it was obtained.

It should be noted that, according to the invention, the quantity X can be measured in all the phases, aqueous and organic, obtained in each of the N rows of partitions in order to improve the experimental accuracy. However, this is not indispensable, only the measurement of the quantity X in all the aqueous and organic phases issued from the last row N of partitions being necessary.

### 4. Mathematical Processing of the Measured Values of X (Steps c) and d) of the Method)

4.1. Definition of the Theoretical Vectors \vec{V}theor(Dₖ):

To define the vectors \vec{V}theor(Dₖ), first, for all the aqueous and organic phases obtained in each of the N rows of partitions, the expected values of X for solutes having y different values of D, noted as Dₖ, are calculated, y being greater than or equal to 2.

These calculations can be carried out by applying the equations (10a) and (10b):

$${X}_{( i + 1 , 2 j - 1 )} ( {D}_{k} ) = {X}_{( i j )} ( {D}_{k} ) ⨯ \frac{1}{( 1 + {D}_{k} ⨯ \frac{{V}_{( ij ) org}}{{V}_{( ij ) aq}} )} \qquad ( 10 a )$$

$${X}_{( i + 1 , 2 j )} ( {D}_{k} ) = {X}_{( ij )} ( {D}_{k} ) ⨯ \frac{{D}_{k} ⨯ \frac{{V}_{( ij ) org}}{{V}_{( ij ) aq}}}{( 1 + {D}_{k} ⨯ \frac{{V}_{( ij ) org}}{{V}_{( ij ) aq}} )} \qquad ( 10 b )$$

which are merely the application of the above equations (9a) and (9b) to the partition of a phase ij (that is to say having a row i and order j in this row) and a synthesis, on the one hand, of the above equations (1a), (2a) and (3a) for the equation (10a) and, on the other hand, of the above equations (1b), (2b) and (3b) for the equation (10b).

In the equations (10a) and (10b):

X_{(i+1,2j-1)}(Dₖ) is the expected value of X for solutes having the value Dₖ and for the aqueous phase issued from the partition of the phase ij,

X_{(i+1,2j)}(Dₖ) is the expected value of X for solutes having the value Dₖ and for the organic phase issued from the partition of the phase ij,

X₍ᵢⱼ₎(Dₖ) is the expected value of X for solutes having the value Dₖ and for the phase

V_{(ij)org} is the volume of the organic phase subjected to the partition ij or the volume of organic solution S_{org} used to carry out the partition of the phase ij (if the phase ij is an aqueous phase), and

V_{(ij)aq} is the volume of the aqueous phase subjected to the partition ij or the volume of aqueous solution S_{aq} used to carry out the partition of the phase ij (if the phase ij is an organic phase).

Thus, applied, for example, to the partition of the aqueous phase φ₂₁ shown in FIG. 2 via a volume of organic solution S_{org}, noted as V₂₁(S_{org}), the equations (10a) and (10b) respectively become:

$${X}_{31} ( {D}_{k} ) = {X}_{21} ( {D}_{k} ) ⨯ \frac{1}{( 1 + {D}_{k} ⨯ \frac{{V}_{21} ( {S}_{org} )}{V ( {φ}_{21} )} )}$$

$${X}_{32} ( {D}_{k} ) = {X}_{21} ( {D}_{k} ) ⨯ \frac{{D}_{k} ⨯ \frac{{V}_{21} ( {S}_{org} )}{V ( {φ}_{21} )}}{( 1 + {D}_{k} ⨯ \frac{{V}_{21} ( {S}_{org} )}{V ( {φ}_{21} )} )}$$

where:

Dₖ is as defined above,

X₃₁(Dₖ) is the expected value of X for solutes having the value Dₖ and for the aqueous phase φ₃₁,

X₃₂(Dₖ) is the expected value of X for solutes having the value Dₖ and for the organic phase φ₃₂,

X₂₁(Dₖ) is the expected value of X for solutes having the value Dₖ and for the aqueous phase φ₂₁,

V₂₁(S_{org}) is the volume of organic solution S_{org} used to carry out the partition of the aqueous phase φ₂₁, and

V₂₁ is the volume of the aqueous phase φ₂₁ subjected to the partition.

As indicated above, the values Dₖ are at least two in number but can be more in number, for example 3, 4, 5, 6, 7, 8 or more in number with:

when they are 3 in number, for example D₁=10⁻¹ (i.e. log D₁=−1), D₂=10°, (i.e. log D₂=0) and D₃=10 (i.e. log D₃=+1), or

when they are 5 in number, for example D₁=10⁻² (i.e. log D₁=−2), D₂=10⁻¹ (i.e. log D₂=−1), D₃=10° (i.e. log D₃=0), D₄=10 (i.e. log D₄=+1) and D₅=10² (i.e. log D₅=+2), etc.

It should be noted that the application of the equations (10a) and (10b) to the partition of the solution S₀ requires knowing the value of X, noted as X₀, for this solution and, thus, having previously measured it.

On the basis of these calculations, it is then possible to establish, for the last row N of partitions, y vectors \vec{V}theor(Dₖ) each having a dimension 2^{N} and the elements of which represent the theoretical contribution of the solutes, which have the value Dₖ, to the quantity X measured in the aqueous and organic phases issued from this row of partitions, i.e. the n^{th} element of the \vec{V}theor(Dₖ) is Vₙtheor(Dₖ)=Xₙ(Dₖ)/X₀ (in %).

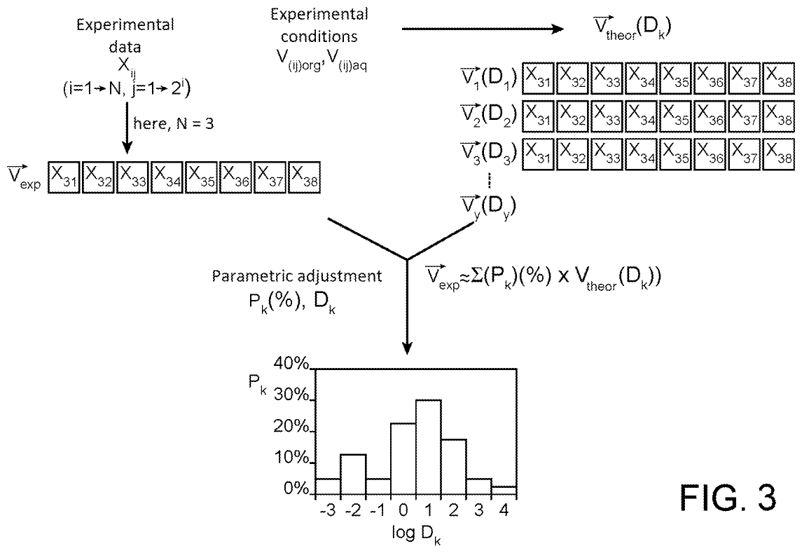

Thus, as illustrated for example on the right side of FIG. 3, the vectors \vec{V}theor(Dₖ) for the sequence of partitions shown in FIG. 2, noted as V₁(D₁), V₂(D₂), V₃(D₃), . . . , V_{y}(D_{y}), each have 8 elements, respectively X₃₁, X₃₂, X₃₃, X₃₄, X₃₅, X₃₆, X₃₇ and X₃₈, each element being assigned, here also, the same index as that affecting the phase, aqueous or organic, to which it corresponds.

4.2. Parametric Adjustment:

As illustrated in FIG. 3, once the vector \vec{V}exp and the \vec{V}theor(Dₖ) have been established, a comparison of the data of the vector \vec{V}exp to a linear combination of the vectors \vec{V}₁(D₁), \vec{V}₂(D₂), \vec{V}₃(D₃), . . . , \hat{V}_{y}(D_{y}) allows to adjust the parameters which are Pₖ (%) on the one hand, and, optionally, Dₖ on the other hand, so that:

\vec{V}exp≈Σ(Pₖ(%)×\vec{V}theor(Dₖ)).

A histogram such as that shown in FIG. 3 can thus be obtained, y being, in the case of this histogram, equal to 8 and the hydrophilicity/lipophilicity being expressed in terms of log Dₖ.

### 5. Validation of the Method of the Invention

An aqueous mixture comprising 3 standard solutes having different lipophilicities (that is to say values of log D), namely benzoic acid, theophylline and benzimidazole, is prepared.

In the experimental conditions used for this validation, the log D of the benzoic acid is equal to −1.24, that of the theophylline is equal to −0.02, while that of the benzimidazole is equal to +1.34.

The total quantity of moles of the solutes in the mixture (i.e. n₀) is approximately 5×10⁻⁴ moles. The pH of the mixture is approximately 7.4 (phosphate buffer: PBS).

A sequence of 3 rows of partitions—as illustrated in FIG. 2—is applied to the mixture by using hydrated n-octanol for the partition of the aqueous mixture and that of the aqueous phases issued from the partitions of rows 1 and 2 (i.e. the phases φ11, φ21 and φ23), and an aqueous solution comprising PBS buffer having a pH of 7 in distilled water for the partition of the organic phases issued from the partitions of rows 1 and 2 (i.e. the phases φ12, φ22 and φ24). The ratio V_{org}/V_{aq} used is approximately ¼.

The concentration of each solute in the phases issued from the partitions of rows 1, 2 and 3 (i.e. the phases φ11 to φ38) is monitored by UV-visible spectroscopy but only with the goal of verifying the proper running of the experiment.

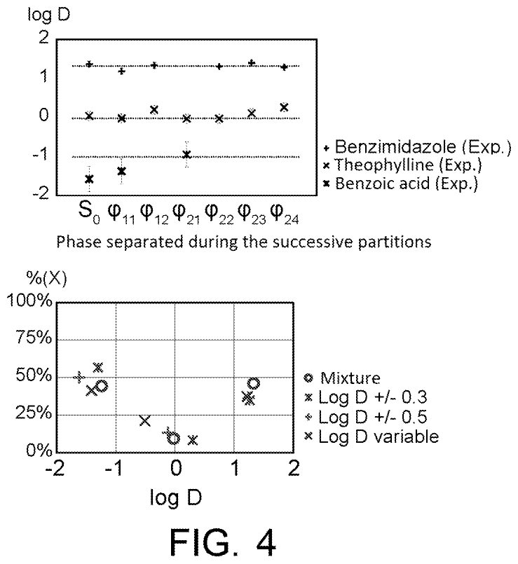

As shown in the left part of FIG. 4, which shows the partition coefficients, expressed in terms of log D, as verified experimentally in each of the rows of partitions and for each of the solutes, the theophylline and the benzimidazole are distributed between aqueous phases and organic phases in a manner sufficiently homogenous for their concentration to be able to be measured in all the phases (except for the benzimidazole in the phase φ31). However, due to its hydrophilic nature, the quasi-totality of the benzoic acid is extracted in the aqueous phases: φ11 then φ21 and φ31. The measurement of its partition coefficient is thus only possible starting from the separation of these three latter phases. The partition coefficients measured individually for the three compounds are close to the theoretical values indicated in dotted lines, attesting to the proper running of the successive experiments.

The results obtained by applying the mathematical processing described in point 4 above are illustrated in the following table as well as on the right of FIG. 4.

This table presents:

the log D of the solutes as verified experimentally, noted as experimental log D,

the number of moles of each of the solutes in the starting mixture as verified experimentally, noted as nₘᵢₓₜᵤᵣₑ,

the log D used to define the theoretical vectors, noted as target log D, and

the number of moles obtained for each solute, noted as nᵣₑₛᵤₗₜ, and obtained by adjustment of the total quantity of the three solutes measured in each phase.

| | Benzoic acid | Theophylline | Benzimidazole | nₜₒₜ₍ₘₒₗₑₛ₎ |
|---|---|---|---|---|
| experimental | −1.24 | 0.01 | 1.33 | |
| log D | | | | |
| nₘᵢₓₜᵤᵣₑ₍ₘₒₗₑₛ₎ | 2.05 · 10⁻⁴ | 4.36 · 10⁻⁵ | 2.12 · 10⁻⁴ | 5 · 10⁻⁴ |
| target log D | −1 | 0 | 1 | |
| nᵣₑₛᵤₗₜ₍ₘₒₗₑₛ₎ | 1.86 · 10⁻⁴ | <10⁻⁵ | 1.66 · 10⁻⁴ | 3.5 · 10⁻⁴ |

In the right part of FIG. 4, the results are expressed in percentages in terms of amount of substance of a solute (nX) relative to the total amount of substance of the solutes in the mixture (nₜₒₜ), noted as P(X)%, according to the partition coefficient, expressed in terms of log D.

The mathematical processing only uses the value of the total quantity (in moles) of the solutes in the phases of the last row of partitions, which is relative to the total quantity of these solutes in the starting mixture.

Various parametric adjustments are tested with a variable number of theoretical vectors (3 or 5) or different values of log D. In each case, the data obtained by the mathematical processing (indicated by signs) is relatively close to the expected values for this known mixture (indicated by circles).

This type of results was reproduced with various ratios of quantities between the solutes, various ranges of concentrations, other solutes, and various types of quantity X (¹⁴C, TOC, etc.), which demonstrates a general feasibility for the method of the invention.

### 6. Evaluation of the Degree of Purity of a Bisphenol a Marked by ¹⁴C and Characterisation of an Impurity

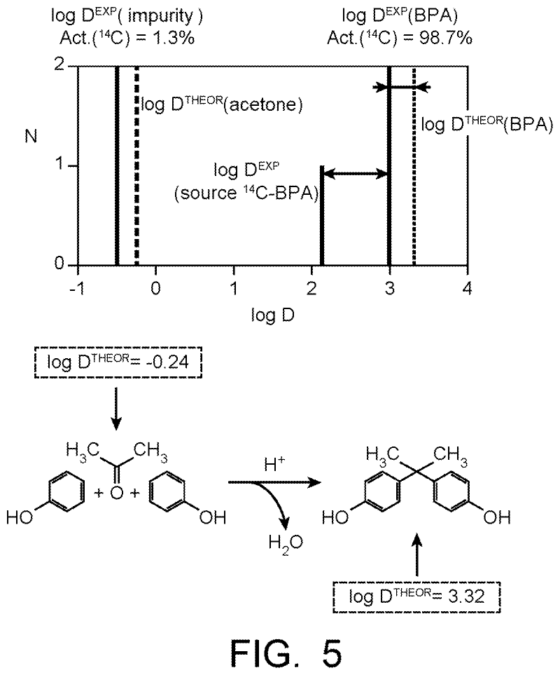

Reference is now made to FIG. 5 which illustrates an example embodiment of the method of the invention for the evaluation of the degree of purity of a synthetic substance and the characterisation of the quantity and of the lipophilicity of an impurity that this substance comprises, which is one of the potential applications of this method.

This example was carried out with a commercial source of bisphenol A (BPA) marked with ¹⁴C, more simply called ¹⁴C-BPA below.

The quantity X is thus the ¹⁴C activity that is measured by liquid scintillation.

As shown in FIG. 5, the measurement of the ¹⁴C activity after a single partition (N=1) of the commercial source of ¹⁴C-BPA, carried out with distilled water and n-octanol, provides a log D, noted as log D^{EXP} (source ¹⁴C-BPA), substantially equal to 2.1 (solid line) and, thus, very different from the log D that the BPA theoretically has, noted as log D^{THEOR} (BPA) and which is 3.32 (dotted line).

Despite a high purity of the commercial source (≈99%), a significant difference is measured with respect to the theoretical lipophilicity of the BPA because of the presence of an impurity having properties very different from those of the BPA: log D (impurity)<<log D (BPA).

A second row of partitions (N=2) is carried out on the phases φ₁₁ and φ₁₂ issued from the first partition (i=1).

The ¹⁴C activity is measured on the 4 phases, φ₂₁ to φ₂₄, issued from this second row of partitions. The results of these measurements are compared using the mathematical processing described in point 4 to the results theoretically obtained for a mixture of two unknown species having different lipophilicities D1 and D2.

As shown in FIG. 5, the following have thus been highlighted starting from the second row of partitions:

on the one hand, a quantity of approximately 99% in ¹⁴C activity, corresponding to the ¹⁴C-BPA with a log D, noted as log D^{EXP} (BPA), of approximately 3 and, thus, close to the log D that the BPA theoretically has, and

on the other hand, a quantity of approximately 1.3% in ¹⁴C activity, corresponding to an impurity having a log D, noted as log D^{EXP} (impurity), substantially equal to −0.5.

It is noted that these results obtained “blindly” agree with the amount of impurity estimated by the supplier, which is approximately 1%, and the presence in the commercial source of ¹⁴C-BPA of possible residues of acetone marked with ¹⁴C. Indeed, as shown in FIG. 5, the BPA is synthesised by reaction of two molecules of phenol with a molecule of acetone, the theoretical log D of which, noted as log D^{THEOR} (acetone), is substantially equal to −0.24.

### REFERENCES CITED

Mondeguer et al., Spectra Analyse 2012, 284, 25-33
Fischer et al., Trends in Analytical Chemistry 2021, 136, 116188

1. A method for characterising a solution S₀ comprising a plurality of solutes, which comprises at least the steps consisting in:
a) subjecting the solution S₀ to a sequence of N rows i of partitions, i ranging from 1 to N and N being greater than or equal to 2, the rows i of partitions comprising:
for i=1, a mixture of a sample of the solution S₀ with a volume of an organic S_{org} or aqueous S_{aq} solution immiscible with the solution S₀ to obtain, after separation of the mixture into two phases, an aqueous phase φ(i=1,aq) and an organic phase φ(i=1,org); and
for i=2 to N:
a mixture of each aqueous phase φ(i−1,aq) with a volume of the solution S_{org} to obtain, after separation of the mixture into two phases, an aqueous phase φ(i,aq) and an organic phase φ(i,org);
a mixture of each organic phase φ(i−1,org) with a volume of the solution S_{aq} to obtain, after separation of the mixture into two phases, an aqueous phase φ(i,aq) and an organic phase φ(i,org);
b) subjecting all the aqueous and organic phases obtained at the row N to a measurement of an extensive and conservative quantity X to obtain an experimental vector \vec{V}exp comprising 2^{N} measured values of X;
c) defining theoretical vectors \vec{V}theor(Dₖ) for y partition coefficient values Dₖ, with y greater than or equal to 2 and k ranging from 1 to y, each theoretical vector comprising 2^{N} values of X calculated for the aqueous and organic phases obtained at the row N and for one of the y values Dₖ;
d) carrying out a parametric adjustment between the vectors \vec{V}theor(Dₖ) and the vector \vec{V}exp to obtain a distribution of the solutes of the solution S₀, which contribute to the 2^{N} measured values of X, as a function of to the y values Dₖ.

2. The method of claim 1, wherein N is between 2 and 20.

3. The method of claim 1, wherein the solution S₀ is an aqueous solution.

4. The method of claim 3, wherein the solution S_{org} is an organic solvent immiscible in water and the solution S_{aq} is water, optionally comprising a ground salt or a pH buffer or both.

5. The method of claim 1, wherein the solution S₀ is an organic solution.

6. The method of claim 5, wherein the solution S_{aq} is water and the solution S_{org} is an organic solvent immiscible in water.

7. The method of claim 4, wherein the organic solvent is a C8 to C12 alcohol, a C5 to C16 alkane, cyclohexane or an aromatic hydrocarbon.

8. The method of claim 6, wherein the organic solvent is a C8 to C12 alcohol, a C5 to C16 alkane, cyclohexane or an aromatic hydrocarbon.

9. The method of claim 1, wherein the quantity X is an amount of substance, a concentration, an absorbance, a fluorescence, a radioactivity or an opacity.

10. The method of claim 1, wherein defining each of the theoretical vectors \vec{V}theor(Dₖ) comprises:
applying to the partition of the sample of the solution S₀, the equations (1a) and (1b):
$${X}_{( i = 1 , aq )} ( {D}_{k} ) = {X}_{0} ⨯ \frac{1}{( 1 + {D}_{k} ⨯ \frac{{V}_{( 0 , org )}}{{V}_{( 0 , a q )}} )} \qquad ( 1 a )$$
$${X}_{( i = 1 , org )} ( {D}_{k} ) = {X}_{0} ⨯ \frac{{D}_{k} ⨯ \frac{{V}_{( 0 , org )}}{{V}_{( 0 , aq} )}}{( 1 + {D}_{k} ⨯ \frac{{V}_{( 0 , org )}}{{V}_{( 0 , aq )}} )} \qquad ( 1 b )$$
applying to the partitions of the phases φ(i,aq), the equations (2a) and (2b):
$${X}_{( i + 1 , aq )} ( {D}_{k} ) = {X}_{( i , aq )} ( {D}_{k} ) ⨯ \frac{1}{( 1 + {D}_{k} ⨯ \frac{{V}_{( i , org )}}{{V}_{( i , aq )}} )} \qquad ( 2 a )$$
$${X}_{( i + 1 , org )} ( {D}_{k} ) = {X}_{( i , aq )} ( {D}_{k} ) ⨯ \frac{{D}_{k} ⨯ \frac{{V}_{( i , org )}}{{V}_{( i , aq} )}}{( 1 + {D}_{k} ⨯ \frac{{V}_{( i , org )}}{{V}_{( i , aq )}} )} \qquad ( 2 b )$$
applying to the partitions of the phases φ(i,org), the equations (3a) and (3b):
$${X}_{( i + 1 , aq )} ( {D}_{k} ) = {X}_{( i , org )} ( {D}_{k} ) ⨯ \frac{1}{( 1 + {D}_{k} ⨯ \frac{{V}_{( i , org )}}{{V}_{( i , aq )}} )} \qquad ( 3 a )$$
$${X}_{( i + 1 , org )} ( {D}_{k} ) = {X}_{( i , org )} ( {D}_{k} ) ⨯ \frac{{D}_{k} ⨯ \frac{{V}_{( i , org )}}{{V}_{( i , aq} )}}{( 1 + {D}_{k} ⨯ \frac{{V}_{( i , org )}}{{V}_{( i , aq )}} )} \qquad ( 3 b )$$
where:
X_{(i=1,aq)} is an expected value of X for solutes having the value Dₖ and for the phase φ(i=1,aq),
X_{(i=1,org)} is an expected value of X for solutes having the value Dₖ and for the phase φ(i=1,org),
X₀ is a value of X measured in the solution S₀,
V_{(0,org)} is a volume of the sample of solution S₀ if the solution S₀ is an organic solution or the volume of solution S_{org} used to partition the sample of the solution S₀ if the solution S₀ is an aqueous solution,
V_{(0,aq)} is a volume of the sample of solution S₀ if the solution S₀ is an aqueous solution or the volume of solution S_{aq} used to partition the sample of the solution S₀ if the solution S₀ is an organic solution,
X_{(i+1,aq)} is an expected value of X for solutes having the value Dₖ and for a phase φ(i+1,aq) issued from the partition of a phase φ(i,aq) or of a phase φ(i,org),
X_{(i+1,org)} is an expected value of X for solutes having the value Dₖ and for a phase φ(i+1,org) issued from the partition of a phase φ(i,aq) or of a phase φ(i,org),
X_{(i,aq)} is an expected value of X for solutes having the value Dₖ and for the partitioned phase φ(i,aq),
X_{(i,org)} is an expected value of X for solutes having the value Dₖ and for the partitioned phase φ(i,org),
V(i,aq) is a volume of the partitioned phase φ(i,aq) or the volume of solution S_{aq} used to partition the phase φ(i,org),
V(i,org) is a volume of the partitioned phase φ(i,org) or the volume of solution S_{org} used to partition the phase φ(i,aq),
whereby, for the aqueous and organic phases issued from the row N, 2^{N} expected values of X are obtained for solutes having the value Dₖ.

11. The method of claim 1, wherein the parametric adjustment comprises determining a set of percentages Pₖ as weighting coefficients such that:
\vec{V}exp≈Σ(Pₖ(%)×\vec{V}théor(Dₖ)).

12. The method of claim 11, wherein the adjustment also comprises adjusting the values Dₖ.

13. The method of claim 1, which comprises expressing the distribution of the solutes of the solution S₀, which contribute to the 2^{N} measured values of X, as a function of the y values Dₖ, in the form of a histogram.

14. The method of claim 1, wherein the solutes are organic solutes.
